(19) United States
(12) Patent Application Publication
(10) Pub. No.: US 2005/0075620 A1
(43) Pub. Date: Apr. 7, 2005

(54) Method and apparatus for the delivery of substances to biological components

(76) Inventor: Yoni Iger, Haifa (IL)

Correspondence Address: Yoni Iger, 113A Einstein Street, Haifa, 34601, IL

(21) Appl. No.: 10/466,796

(22) PCT Filed: Jan. 22, 2002
(86) PCT No.: PCT/IL02/00056

(30) Foreign Application Priority Data
Jan. 26, 2001 (IL) 141123

(51) Int. Cl. A61M 31/00

(52) U.S. Cl. 604/500; 604/68; 604/22

(57) ABSTRACT

The invention concerns a method and device for needle-less delivery of substances into or through natural or artificial biological components such as membranes, organelles, cells, tissues, organs, or creatures, by exposing the said biological components to accelerated substances wherein high impact mechanical movement over short distance is used to create acceleration of substances so to drive substances into or through said natural or artificial biological components, while isolating the biological component from the driving force. The mechanical movement is preferably created by an ultrasonic member having a high repetition rate, and the space between accelerating element and biological target is preferably composed of low density compound. The delivery device can be provided with a unit for supplying substance to be delivered, to the mechanical accelerating element. The device can be constructed either as delivery device for superficial tissues, or as an endoscopes laparoscope-like or catheter-like device for delivery in minimally invasive procedures.

### FIELD OF THE INVENTION

The present invention concerns a method, device and system for the delivery of accelerated substances, soluble or particulate, to and through biological components such as membranes, organelles, cells, tissues, organs or creatures, using ultrasound as a preferred accelerating agent and while isolating the biological component from the driven force.

### BACKGROUND OF THE INVENTION

Needle less delivery of substances such as mechanical stabilizers, drugs, nutrients, gene-carriers, vaccines or metabolites, either as particles or in solution, into natural or artificial biological components, is often faced with difficulties due to mal-penetration attributed to the barriers functioning against undesired penetration of foreign components. Also topical delivery to internal zones of biological components is faced with difficulties associated with mal-permeability of biological component.

Ionphoresis, high pressure injection, or ultrasound are among the techniques developed for the facilitation of efficient and safe administration of substances into biological components, mostly of superficial zones.

For example, ultrasound is used for facilitation of transport of various compounds across tissues, typically skin (Mitragotri, M., et al., Science, 269:850-853 (1995)).

The ultrasonic delivery was improved by using ultrasound in conjunction with chemical permeation enhancer and/or iontophoresis (U.S. Pat. No. 5,231,975). Other methods use ultrasonic waves to excite the local nerves, thereby to open the epidermal/dermal junction membrane and the capillary endothelial cell joints, which enables the transfer of drugs through the skin and into the blood stream (U.S. Pat. No. 5,421,816) or delivery through two pulses where the first one enlarges the intercellular spaces and the second one enables delivery thereof (PCT/IL97/00405). Significant problem of the conventional ultrasound, ionphoresis or chemical assisted delivery is that during the process the biological component is constantly exposed to the driven stimulus, such as irradiation.

It is also desirable to deliver into biological components relatively large amounts of solutions, or complex particles. State-of-the-art ultrasound-facilitated administration methods are unsuitable for administration of said solutions or complex particles, since application of ultrasound pulses, sufficient to drive a small amount of small-sized molecule through a tissue is insufficient to drive large amounts or to drive those complex particles through tissues or biological or artificial membranes. Increase of the duration, or intensity, changes of frequency or of the ultrasound pulses to levels which are presumably sufficient to drive the large amounts of solutions or particles through the tissue or cell membrane in one operation, or a serial of repeated operations, has not been reported probably since it results in irreversible damage to the tissue and in significant cell-death. Similarly, irreversible damage occurs in non-biological membranes of e.g., polyethylene or elastomer (for example those used in implants), when increased intensities or durations of ultrasound irradiation have been used.

Other devices perform delivery of compounds by employing a pressure enforced from compressed gas reservoir or by gas spring to create sufficient pressure enabling pushing of medication through e.g., the skin tissue (U.S. Pat. No. 6,096,002). Significant problems here include the need of high-pressure gas reservoir, moving pistons, or gas release, which restricts application to only certain external tissues.

At times, it is desirable to deliver into biological components substances in the form of solutions, or particles, without accompanied energy delivery to the biological component, without gasses flux or moving pistons. It is also desired to do substances administration regardless of their molecular weight, ionic condition, size or polarity.

It would have been highly desirable to provide a method for a single as well as high repeatability delivery of wide variety of substances to natural or artificial biological components, either superficial or internal, utilizing driving force, while isolating the driving force from the biological components, therefore minimizing the damage to the tissue or cells. It would have further been desirable to provide an ultrasound facilitated method for delivery of solutions or complex particles having a relatively large size and without employing pressure to the compound to be delivered, nor ventilation or energy delivery to the treated area. It is the object of this invention to provide a method and device for multi purposes intra tissual delivery, which avoid limitations of current technologies and reduce their possible side effects.

### SUMMARY OF THE INVENTION

In view of the above, the present invention provides a novel method and device allowing the delivery of substances to, into or through biological components that are part of or an entire biological entity. Said biological components might be membranes, organelles, cells, tissues, organs or creatures. This, in accordance with the invention, is achieved by utilizing an ultrasound stimulus, or other energy source capable of producing high acceleration rate over short distance and at high repeatability, to accelerate the substance to be delivered via low density medium and in the direction of the biological component. By accelerating the substance attached to an ultrasonic vibrating element, or other high accelerating means, in a low density medium, it has been found that substances continue to move in direction of acceleration and it was possible to deliver substances while affecting only the attached substance and isolating the biological component from the energy source, therefore enabling substance delivery without energy delivery to the biological component and without causing it energy related damage. The method for the delivery of substances to biological compounds shall be preferably performed via low density medium such as gas or vacuum. The delivery does not involve any gas streaming or moving parts, and is applicable also to internal tissues.

The method, in accordance with the invention, comprises the step of exposing the substance to be delivered to a high amplitude ultrasound stimulus, being such as to accelerate the ultrasound attached substance, kept at certain distance from the biological component, via low density medium and in the direction of the said biological component. Surface of the ultrasound generating element, might be covered by compounds capable of reducing the surface-tension, therefore enabling easy release of substance to be delivered. Due to the distance filled with low density medium between the energy source and the substance to be delivered on one hand, and the biological component on the other hand, said energy created by the accelerating means is markedly attenuated in the low density medium and essentially do not reach the target biological component. On the other hand, the substance to be delivered is accelerated with minimal friction during delivery and eventually at least part of it reaches and penetrate the biological component. The disconnection between the accelerating agent, for instance the ultrasound, and the biological component, as well as the non pressurized procedure, enable delivery without causing damage to the bulk of said biological component, at high repetition rate and also to internal zones of biological components, as is below explained.

The method of the present invention may be used for therapeutic and cosmetic purposes, as well as for diagnostic and experimental purposes, according to the type of substance to be delivered, and the relevant biological component.

By one non limiting embodiment, the substances to be administered may be soluble substances such as various medicaments for therapeutic treatment, anti ageing agents for prevention purposes, toxic compounds for controlled degeneration, growth factors hormones or interleukins for the initiation or cessation of processes, amino acids or proteins or substrate elements to be resources for macromolecules or processes, macromolecules such as DNA molecules or their fragments, for the purpose of gene therapy or genetic manipulation, various dyes for the purpose of diagnosis inside cells, or within a tissue, substances for local anesthesia, substances for topical destruction of biological component, substances for the reduction or acceleration the activity of biological component or of any sub biological component including infectious agents, substances for changing the mechanical or chemical properties of a biological component or any of it's subunits, and the like.

By yet another non-limiting embodiment, the substances to be administered are complex particles. The term “particles” or “complex particle” refers generally to a particle having the size of at least 1 nm ranging to tens or hundreds of microns which is usually composed of a single type of molecule, or alternatively several types of molecules. The complex particles are essentially insoluble in the medium in which they are carried. Examples of complex particles are granules of toxic compounds, sensitizers or radioactive compounds, attenuated or killed disease-causing agents or parts thereof such as bacteria, virions, fingi, protozoa or parasites administered for the purpose of vaccination; plasmids containing DNA to be inserted for the purpose of gene-therapy or genetic manipulations; nano-particles with genes or DNA vaccines, nanomachines, nuclei of gametes administered into oocytes for the purpose of fertilization; particles impregnated with medicaments capable of releasing them at a slow rate to the surrounding tissue for the purpose of therapy or controlled immune reduction; particles containing compounds that were coated with a protective coating, for example, in order to form particles having different solubility, to prevent oxidation, to prevent a hygroscopic effect, to increase resistance to heat or to protect the contents of the particle from biological effects (such as degradation); particles comprising a biologically compatible dye for the purpose of tattooing, as for example, in the case of permanent makeup; particles comprising a detectable marker for the purpose of diagnosis, and the like.

According to another non limiting example, particles might be also inert or other compounds of particular characteristics, accelerated towards biological components at high acceleration rates, from short distance and at certain angle, and affecting by disconnecting, causing destruction and removal of sub-components or layers of said treated biological components.

Particles or solutions might be also active or inert compounds used for mechanical support or stabilization of biological components. Active or inert particles might be also used for paving of biological components.

The “biological component” to which the substances are administered, can be any type of membranes, organelles, cells, tissues, organs or creatures. Biological component might therefore refer to eukaryotic or prokaryotic cells, or their sub-components, including cells cultured in a medium. Biological component might further refer to epithelial tissues which may be keratinized epithelial tissues such as skin, or moist-epithelial tissues, for example, the epithelium lining the eyes, digestive tract, respiratory, or reproductive systems. The tissue may also be the moist epithelial tissue covering aquatic creatures such as fish, crustaceans or mollusks at different stages of rearing, including embryonic ones.

The term “biological component” might also refer to artificial components, being parts of, or replacing parts of, or assisting the activity of, or used as mechanical support for, or used for releasing substances to or through natural membranes, organelles, cells, tissues, organs or creatures. Therefore the term biological component might refer also to elastomer compounds which form part of an implant, or to artificial skin which has been constructed for replacing damaged skin area, to encapsulated cells or artificial tissue constructed for slow release, or similar artificial components, as the case might be.

The substance accelerating stimuli, created by ultrasound or any other accelerating mean, are applied when the relevant biological component is not in contact with the accelerating stimuli mean, nor in contact with any liquid medium or gel coupling medium that form a bridge between the biological component and the accelerating mean, but remains isolated from the accelerating stimuli when that is being performed. The medium between the stimulating element and the biological component is essentially composed of an ultrasound isolation medium, such as gas or vacuum, and not of ultrasound coupling medium.

The substance to be administered shall be acoustically coupled to the stimulating element at least during part of the operation period. That is to say that coupling might be on permanent or temporal basis. Temporal coupling might be achieved for instance, during at least part of cycle of the acceleration of the vibrating element towards the substance. The substance may be present in liquid, gel, paste, powder, pellet, solid strip and the like. It might be composed of homogenous materials or alternatively of different compounds mixed together close to the vibrating element before the delivery, or mixed in the space between vibrating element and biological component during delivery, or mixed in the biological component after the delivery.

According to non-limiting embodiment, more then one compound is delivered to gain the desired effect. This according to the invention can be performed by having same accelerating rates to the different compounds, or alternatively performing different accelerating rates due to substance weight or size, or by delivery from more than one vibrating element and more than one substance-supply sub-devices. When more than one stimulus is being given, the stimuli may be applied one after the other or simultaneously.

The specific parameters of the stimulus, capable of driving the administered substances into or through said biological components, should be determined empirically, depending among other things on the nature of the biological component, on the nature of the administered substance and on the parameters of the accelerating mean. However, at least one stimuli composed of at least cycle portion shall be given to deliver unit of substance.

Generally speaking, the driving stimulus has the following parameters: Frequency: At least 1 Hz; Preferably 10 Hz to 30 MHz, more preferably 10 kHz to 3 MHz, most preferably, 20 kHz to 100 kHz. Duration: At least quarter of cycle; Therefore at least 0.025 sec. or 0.75×10⁻⁷ sec for 10 Hz or 30 MHz respectively. Amplitude: At least one micron; Preferably 10 to 10,000 microns, most preferably 20 to 200 microns. Intensity: 0.0001-10,000 W/cm², preferably 0.1-100 W/cm², most preferably 3-50 W/cm². Under preferred embodiment, the ultrasonic force is used to cause acceleration of substance to be delivered to the site of administration in the biological component.

It shall be understood that also in the ultrasonic range of frequencies, certainly below it, also other means might be used to create the acceleration force. Such means might include any means that can produce high acceleration rates, over short distance of movement and at high repeatability, for instance sonic speakers, electromagnets, motors, motor-coupled ex-centers, liquid-containing pistons and the like.

Generally speaking, the acceleration rate can be determined as a=ω²Asin(ωt), where A is the amplitude of movement in meters, and ω=2πf, where f is the frequency in Hz. For example, when the frequency is 20 kHz, and the amplitude of vibration 100μ (100×10⁻⁶ m), and maximum acceleration is achieved (i.e., sin(ωt)=1) then acceleration of 1,570,000 m/sec² or about 160,000 g is achieved and can be utilized for delivery. However, it should be appreciated that there exists a reversal proportion between the parameters. For instance, when higher frequency is used, the amplitude can be reduced to achieve similar acceleration rate. At times that ultrasonic transducer is used to create the acceleration stimulus, the high amplitude is essentially created by amplification of the original amplitude of the piezoelectric crystals, or other source, using a horn or tip preferably designed to be in resonance under operation conditions.

Occasionally, biological component might pass pre-delivery treatment to increase their susceptibility and the efficiency of delivery. Said treatment might for example include adherence of cells which are the target of delivery under in vitro conditions, or removal of superficial layers of tissue, such as mucus secretions or keratinized epithelium. According to one non limiting embodiment, the pretreatment might be performed with the same delivery device, operated for instance under streaming or cavitation mode. During such pre-treatment process, a coupling medium shall essentially be present between the accelerating element and the biological component. At times, pre-treatment might be carried out also having gas medium between the driving element and the biological component, and acoustic pressure can be performed to achieve desired pre-treatment effect Pre treatment, however, might be carried out also using other methods and devices, not part of the current invention.

At times, biological components might pass post-delivery treatment. Said post-delivery treatment might be carried out using the same delivery-device, or methods and devices not part of the current invention or their combination. According to non limiting example, post-delivery treatment might include activation when the delivered agents are irradiation-activated substances. According to one embodiment, the agents might be activated by ultrasound, for instance sonosensitizers such as dimethylformamide, N-methylformamide, or dimethylsulfoxide, or activated by light or other energy modalities after delivery. Substances might be also active in nature, for instance radioactive agents, or activated before, or during their delivery due to ultrasound, light irradiation or other stimuli. According to this example, activated substances are being delivered and effect is performed already during their penetration route so that essentially all the region from the site of administration to the region where the substances reached is essentially destroyed.

According to yet another non-limiting example, post treatment might include controlled degeneration of at least portion of biological component. According to one embodiment, said degeneration is performed after delivery of substances such as vaccines, so creating a biological reservoir for the slow release of substance during normal process of phagocytosis and absorbance of the degenerated tissue. The degeneration might for instance be performed by allowing the accelerating device to touch the biological component for a short period of time, causing friction and degeneration.

The present invention also concerns a system for use in the above method. In the following the invention will be further illustrated with reference to some non-limiting drawings and examples.

### BRIEF DESCRIPTION OF THE DRAWINGS

FIG. 1 shows a schematic drawing of the ultrasound delivery device of the invention: 1A before operation and 1B during delivery process.

FIG. 2 shows a schematic representation of a multi-lobed delivery device used in the system of the invention: 2A before operation and 2B during delivery process.

FIG. 3 shows another schematic drawing of a multi lobed device having another substance-supply unit: 3A before operation and 3B during supply of substance to be delivered.

FIG. 4 shows another schematic drawing of delivery device having another actuating mechanism.

FIG. 5 shows a schematic drawing of a laparoscope-implanted delivery device.

FIG. 6 shows another schematic drawing of a laparoscope-implanted delivery device, having particular substance supply method and component.

FIG. 7 shows a schematic drawing of delivery device, having another substance supply method and a concentration element.

FIG. 8 shows another schematic drawing of a delivery device, having another substance supply method and a dispersion element.

FIG. 9 shows yet another schematic drawing of a lateral delivery device.

### DETAILED DESCRIPTION OF THE INVENTION

In the device for substances delivery to biological components, high accelerartion rates are utilized to enforce substances delivery from accelerating element to or through biological component, while isolating the biological component from the driving force. The energy is essentially utilized to push the substances, and it essentialy does not reach and consequently is not absorbed in the biological component. The resultant delivery is therefore free of side effects related to energy absorbance and can be performed in superficial as well as in deeper parts of biological components.

A delivery device, in accordance with the invention, functions to deliver solutions as well as particles, yet essentially does not permit the energy to be delivered, therefore prevent the biological component to be affected by the delivery force. As will later be explained, this result is accomplished by drawing continuous vibration and heat away from the biological component.

The delivery system of the invention generally comprises a control unit, a single or a multi-frequency signal generator, a signal amplifier, a matching unit and at least one transducer which may be attached to an amplitude increasing devices, such as resonator or resonating tip. These elements which increases the amplitude, actually increases also the acceleration rate, having small displacement. The phenomena essentially occur at the distal part of said resonating tip, yet might occur also in other locations according to the planning.

At times other means to create high acceleration rate over small displacement might be used either with the ultrasonic driving force or together with other means, or as stand alone means. The high amplitude element is however encased in a housing. The system further comprises substance to be delivered, which may be brought manually or automatically to the accelerating edge by supply elements. The system is provided by a spacer enabling keeping the biological component at certain distance from the accelerating element during operation. This distance might be changed, increased or decreased. At times, for instance during post treatment procedure, distance between resonating element and biological component might be reduced to zero. The system is most preferably also provided with vacuum element, or gas delivery system, to apply low viscosity medium between the accelerating element and the biological component. The vacuum device might be used also for the suction of the non delivered substances from site of delivery, for instance at the end of treatment. The system might be provided also, by element for supply of media for pre-treatment, means for activation of delivered substances, or other means as the case might be.

It is important to note, however, that the system can be operated as stand alone device, for instance during external procedures; It can be further operated as an add-on to other devices, for instance by implanting a high-rate accelerating device, such as resonating transducer, at the distal part of plunger of a gas-spring needle less injection device, or other delivery device, thereby enabling higher acceleration rates to the substances; It can be further operated in laparoscope devices, for delivery to internal biological components, in conjunction with diagnostic element for monitoring the delivery-device location.

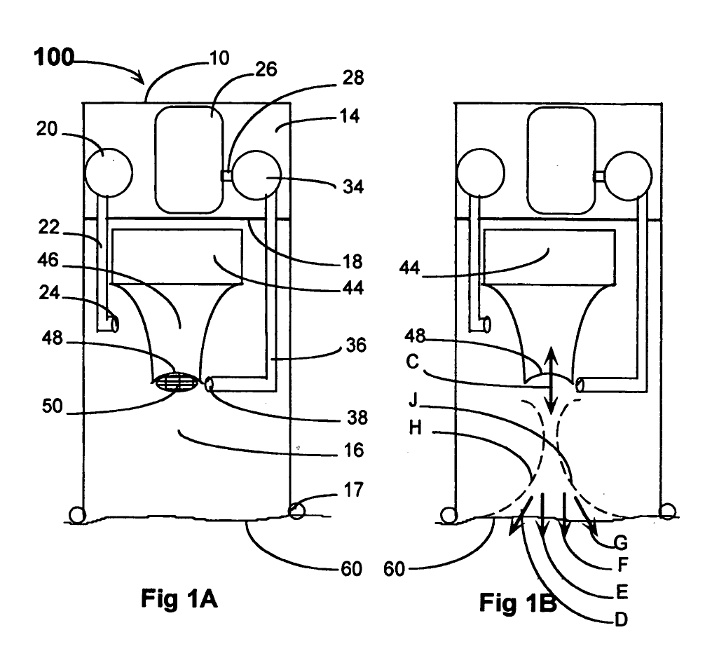

A conceptual model of the delivery device in accordance with the invention, is shown in FIGS. 1A and 1B. The system operated by electricity, is composed of control unit, a signal generator, a signal amplifier, and matching unit (not shown) which are connected to the said delivery device. The device 100 is encased in housing 10. It contain a piezoelectric element 44, transforming the electrical signal to mechanical displacement, and a tip 46 enabling high amplitude at distal end 48, with still the original high repetition rate. The device contain substance reservoir 26, linked by tube 28 to pump 34 which delivers said substance via tube 36 and opening 38 to distal end 48. Element 50 represents the substance accumulated at distal end 48 of the tip 46. The device is separated to two compartments, by septum 18. Vacuum pump 20 is having suction activity of air and debris via opening 24 and tube 22. At the end of the suction activity, the air pressure at space 16 of the septum is lowered. Leakage of gasses from the surroundings is prevented by attaching housing 10 having suction rubber 17 in a shape of circular rim at it's distal open end, to surface 60 of the biological component. The air pressure at space 14 at the other side of septum 18 can remain without change.

In practice, as can be shown in fig 1B, during activation piezoelectric element 44 transform the electrical signal to mechanical displacement. Maximal amplitude of displacement is achieved at the distal end 48 of the tip, which is displaced and accelerated in the general direction of arrow C. The rapid displacement enforces the substance previously attached to end 48 to be detached and move forward. The acceleration and accompanied forces push the substance in the general direction of arrows D,E,F and G between schematic broken lines H and J, into and through surface 60 of the biological component.

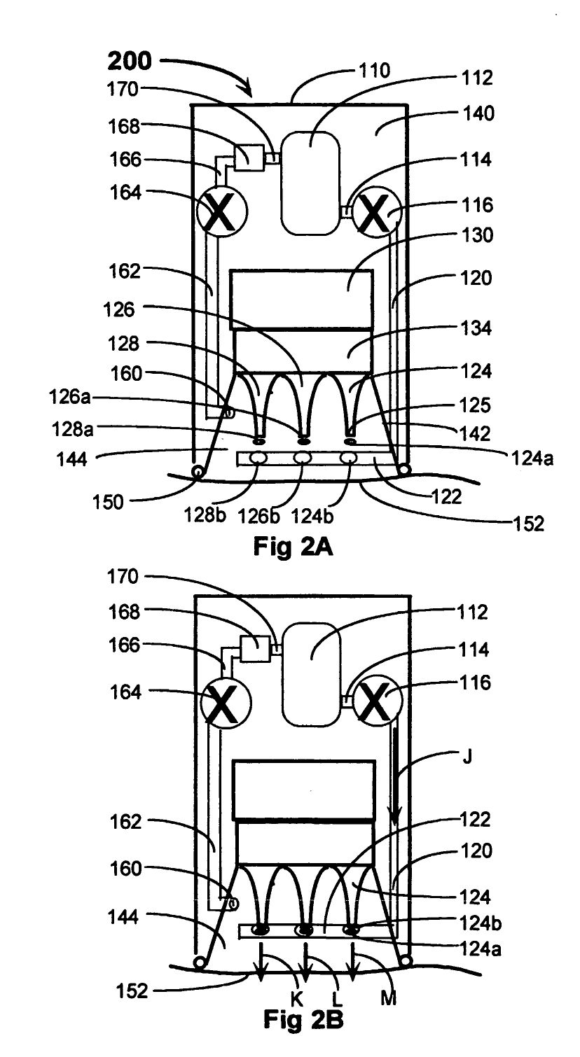

According to the example given is FIGS. 2A and 2B a multi lobed device 200 might be used. The device in housing 110 attached to surface 152 of biological component via suction rubber 150, is composed of a piezoelectric element 130, guiding horn 134 and distal end composed of several tips 124, 126 and 128. Attached to, or alternatively part of, the distal ends of the tips, for instance end 125 of tip 124, are small units 124a, 126a and 128a, capable of absorbing liquids (for instance firm sponge). Substance is supplied from reservoir 112, to tube 114 and pump 116, via tube 120 in space 140, and further to tube 122 in space 144. Tube 122 has holes 124b, 126b and 128b, in the same number of the tips, and in a location compatible to said tips 124, 126 and 128, respectively. Occasionally, different substances might be delivered from different reservoirs to different tips.

When pump 116 is activated, holes 124b, 126b and 128b become filled with substance to be delivered. Suction activity performed by vacuum pump 164, via opening 160 and tube 162, reduces gas content in compartment 144. Suction activity might also facilitate the supply of substance from reservoir 112 to the general direction of space 144, separated from space 140 by septum 142. The supply is in the general direction of arrow J in FIG. 2B. It shall be noted that excess of substance in space 144 is carried out via the suction activity into opening 160, tube 162, and via pump 164 to tube 166, filter 168 and back to the reservoir via tube 170. As demonstrated in FIG. 2B, during operation distance between tips, for instance tip 124 and its absorbing unit 124a on one hand, and holes, for instance 124b on the other hand, is diminished. Supplied substance enters absorbing unit 124a, and similarly enters 126a and 128a, and the accelerating tip 124, and similarly 126 and 128, deliver substance towards surface 152 in the general direction of schematic arrows K,L and M.

It shall be appreciated that as pre-treatment, space 144 might be filled with gassed distilled water, and cavitation performed using irradiation via tips 124,126 and 128. At the end of said pre-treatment water shall be pumped out via suction hole 160.

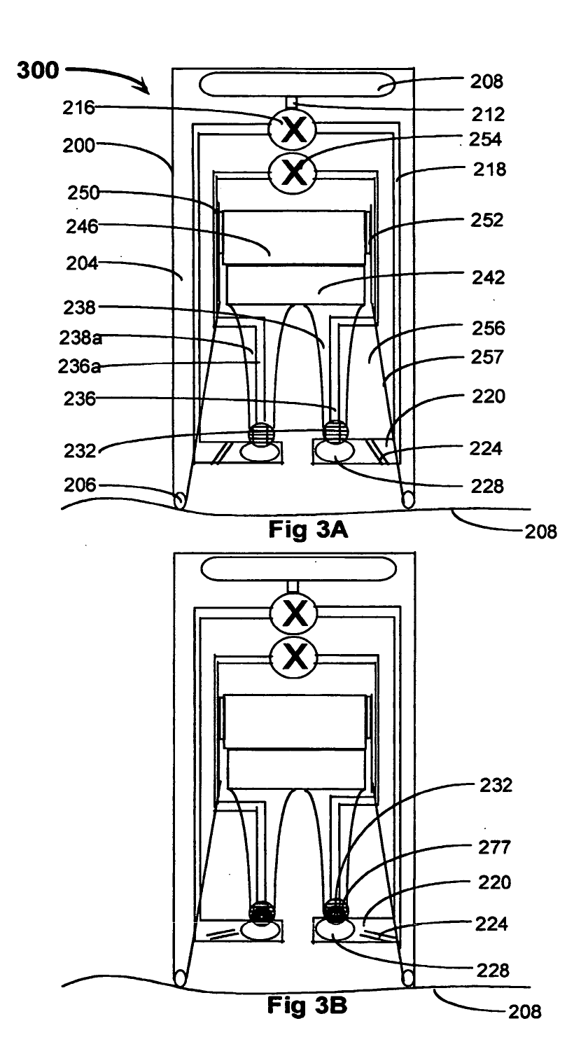

FIG. 3 schematically describes device 300 of the invention. Delivery device is encased in housing 200 attached to the biological component via rubber ring 206. Piezoelectric transducer 246 is coupled to tips 238 and 238a via coupling horn 242. The transducer is attached to inner wall 257, separating between spaces 204 and 256, via attachment unit 252 that also prevents leakage of gasses between spaces. Substance, for instance in the form of particles, is kept in reservoir 208, from where it can be delivered via tube 212 and pump 216, to tube 218 and tube compartment 220. Said tube compartment 220 has hollowed area 228 and a valve 224. At the distal end of each tip 238 and 238a, a lattice 232 is present. The tips are partially hollowed; From lattice 232 tube 236 and 236a run, via tube 250 into vacuum pump 254. During supply of substance, or at certain synchronization with supply of substance, suction activity of pump 254, opens valve 224 to allow particles to be supplied to hollowed area 228. Substances then are accumulated at lattice 232 and form aggregate 277. Ultrasonic pulse will deliver particles of aggregate 277 towards and into surface 208 of biological component.

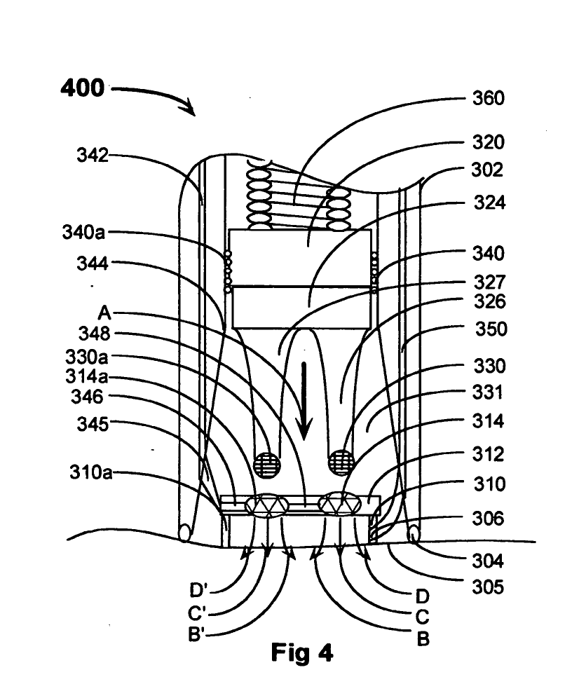

System 400 of FIG. 4 describes another non limiting example of a device. According to this embodiment, inside housing 302 attached to biological component 305 via rubber ring 304, the accelerating element is attached at its proximal end to spring 360. The accelerating element, composed of proximal ultrasonic transducer 320, guiding horn 324 and tips 326 and 327, is attached to inner wall 344 via rings 340 and 340a, that serve also to prevent gas transfer between compartments. However, operation might be performed also when similar pressure exists in the different compartments. Certain degree of vacuum of space 331 is carried out by suction activity of vacuum pump (not shown), via tube 350 and opening 306. Supply of substance is carried out from reservoir and pump (not shown) via tube 342, passing wall 344 via tube 345, via tube 346 into lattice or absorbent element 314a (for particles or solutions respectively) and further via tube 348 to lattice or absorbent 314. Both 314 and 314a, and the interconnecting tubes, are located on stab 312 kept at certain distance from surface of biological component 305, by legs 310 and 310a.

During actuation, releasing of spring 360, causes movement of the accelerating element in the general direction of arrow A, towards lattice/absorbent 314 and 314a. When distal parts 330 and 330a having high accelerating rate, of accelerating element, touches area 314 and 314a, substance located in said 314 and 314a is accelerated and delivered towards surface 305 of biological component in the general direction of schematic arrows B,C,D,B′, C′ and D′. The whole inner construction might be also circular, for instance circular shape of tip, circular shape of lattice or absorbent and so on. The impact of contact between vibrating edges 330 and 330a, and elements 314 and 314a on the other hand, causes the accelerating element to move backwards with spring 360, new substance is applied to 314 and 314a, and the procedure is being repeated. At the end of procedure, as post-treatment, edges 330 and 330a can be vibrated while attached to surface 305, thereby causing local destruction at surface 305. It will be followed by slow release of substances, where the biological component itself serves as reservoir. Post treatment might also for instance include activation of sonosensitizers, previously delivered to biological component.

It shall be appreciated, that with few modifications, the schematic device described in FIG. 4 might be also utilized as add-on that significantly improves performance of, for instance gas spring actuated injection devices, of for instance Medi-Ject Cooperation, or Bioject, Inc., Genesis Medical Technologies, Inc., Weston Medical LimitedRymed Technologies, Mycone Dental Supply Co. Ferton Holding and the like. According to a non-limiting embodiment part of the present invention, at the front edge of piston, or gas releasing orifice, or elsewhere, a high accelerating agent such as ultrasonic element of high frequency and relatively high amplitude (preferably tenth of millimeter), or edge of ultrasonic vibrating tip, is placed to further accelerate substances, in addition to the spring or gas pressure originally used.

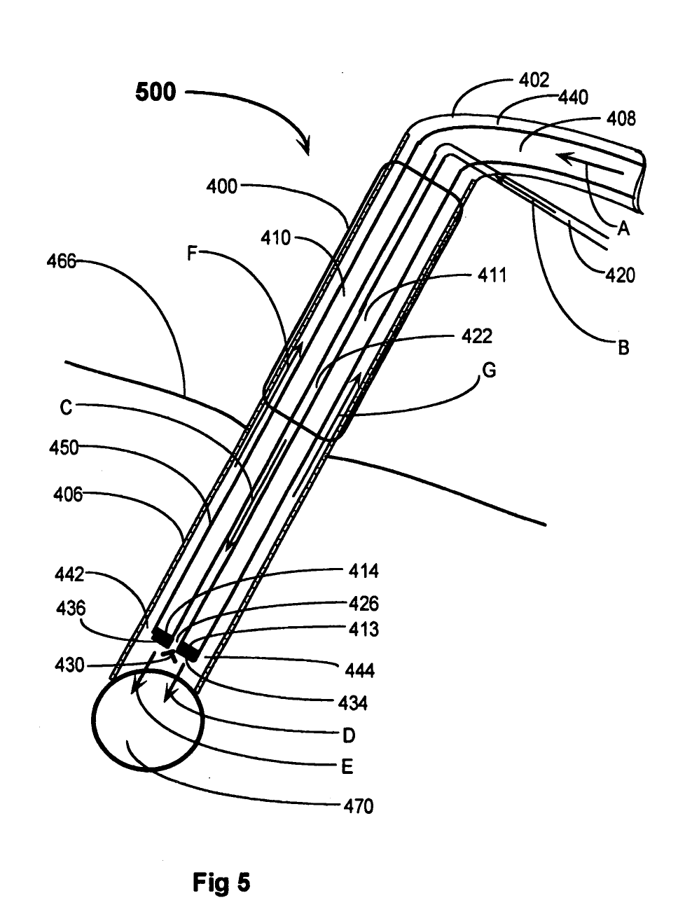

FIG. 5 schematically describes device 500 for delivery to internal tissues. The system is composed of control unit, signal generator, amplifier, matching unit and transducer, as well as possibly increasing amplitude element such as tip, all of which are not shown. Movements created by the transducer (not shown) are transferred to the treatment device, via a wave guide 408 in the general direction described by arrow A. The wave guide is designed so its dimensions till ends 413 and 414 enable movement at resonance of distal ends 413 and 414. Space 440 between wave-guide and wave-guide sleeve 402 is preferably under certain vacuum conditions, as will be further explained below. Tube 420 enters said wave-guide at a point which is preferably a point of minimal movement, for instance zero point. Tube 420 supply the substances from a reservoir (not shown) in the general direction of arrow B, and further via continuation tube 422 in the general direction described by arrow C. Tube 422 might be a hollowed area of a rounded wave guide, and then parts 410 and 411 actually refer to two sides of a cylinder, but it can be also a channel between two separated (and for instance flat), wave guides 410 and 411. Supplied substances leave tube 422 via opening 426, reaches reflecting valve 430, and are reflected and accumulated in lattice, or sponge, 434 and 436 (for particles or liquid), which again might be two sides of a cylindrical component.

Certain degree of vacuum is created by a pump (not shown). Suction of air, cellular debris or excess of substance is performed from the area between delivery device 500 and internal biological component 470. Suction is performed via the spaces 442 and 444, between device laparoscope-wall 406, and wall 450 of accelerating element of the wave-guide 410 and 411, in the general direction of schematic arrows F and G. The supply of substances might be via pushing them with a pump via tube 420, but also by suction activity from the reservoir.

During activity, while held by handle 400, and while wall 406 serves as laparoscope guide, delivery device is inserted via surface 466 to desired location, for instance organ 470. Certain degree of vacuum, according to the needs is performed so that at least space between elements 436 and 434 on one hand, and organ 470 essentially contains no liquid or cellular debris. Substance is delivered to be accumulated in elements 434 and 436. Accelerating element is operated to create high amplitude repeated movement of ends 413 and 414 of the wave-guide. Substance accumulated in 436 and 434 is accelerated towards and into organ 470 in the general direction of arrows D and E. According to non-limiting example, organ 470 might be a tumor and the substance to be delivered composed of Tumor Necrosis Factor.

Generally speaking, the suction activity and the accompanied reduction of air pressure, aim at increasing the isolation capabilities of the space between biological component and accelerating element, and concomitantly to reduce friction of the accelerated substance and air. It shall be noted, however that procedure can be performed also via gasses, and other media.

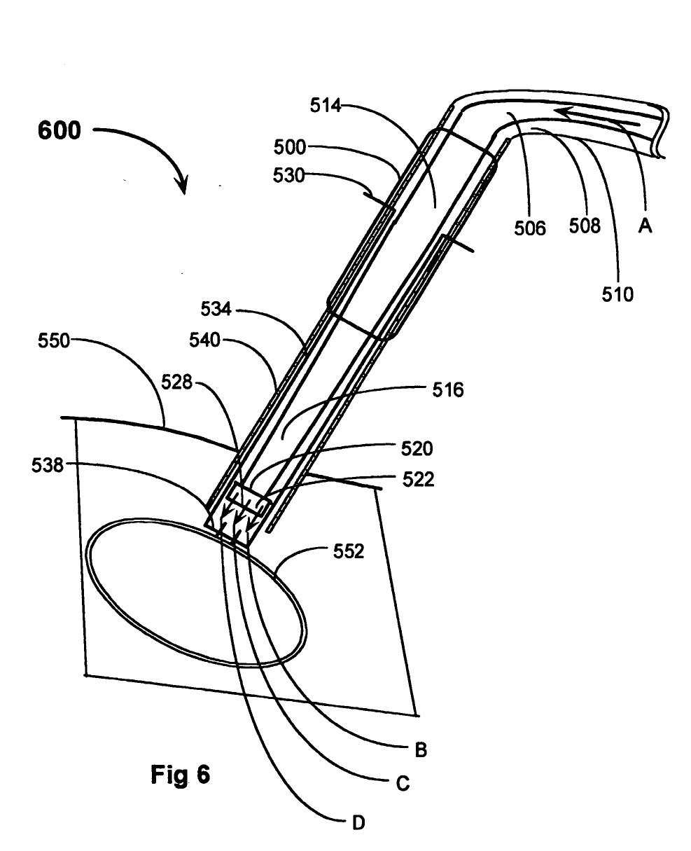

FIG. 6 schematically describes device 600 for delivery to internal tissues. The system is composed of control unit, signal generator, amplifier, matching unit and transducer, all of which are not shown. Movements of high accelerating rate, created by the transducer (not shown) are transferred in the general direction of schematic arrow A, via wave-guide 506. Wave guide 506, is mechanically isolated from sleeve 510 by space 508, containing gas or slight degree of vacuum. Similar isolation exists also between the other accelerating components, such as tip 514 or delivery distal end 522, and laparoscope cover 540. The tip has larger cross section in area 514, and lower cross section closer to the distal end, at area 516, and therefore amplitude of movement is increased under the same frequency and acceleration rate is increased. The whole device is designed for activity under resonance, so that area of maximal movement, and maximal accelerating rate, is at distal end 520 of the tip. The space between tip end and lattice wall 528 contain the substance to be delivered.

At times, a device where the substance fills the wave-guide, might be used. In such case, a liquid substance medium, or gel with appropriate substance to be delivered, is the content of at least last portions of the wave guide, including for instance area 506, 514 and 516 and with continuity to the area between tip end 520 and lattice 528, for instance via openings in tip end 520. Alternatively, the wave-guide may be composed of solid material, or liquid not relevant for the delivery, and for instance only the space between 520 and 528 with said substance to be delivered.

During operation, the device held in handle 500, is inserted via surface 550 of biological component till target 552, having laparoscope wall 540 as guiding element. During insertion, hollowed grid-like end 538 is in same line as end of wall 540. When laparoscope wall reaches target 552, insertion stops. At this stage, pushing of sub-handle 530, transfer further movement of hollowed grid-like end 538, via walls of cylinder 534. Movement of grid-like end 538 might press a bit target 552, but in addition it increases the distance between lattice 528 on one hand, and hollowed grid-like 538 and target 552 on the other hand. This increase of distance is performed and concomitantly, or shortly after and essentially before the increased distance is filled by liquids, waves are emitted and high acceleration is performed to affect tip end 522. It further accelerates substance via lattice 528 which might have larger area than tip end 522, and via hollowed grid-like end 538, into target 552, in the general direction of arrows B,C and D. The ultrasonic path might be also constructed in a different way, so having for instance the ultrasonic transducer in handle 500.

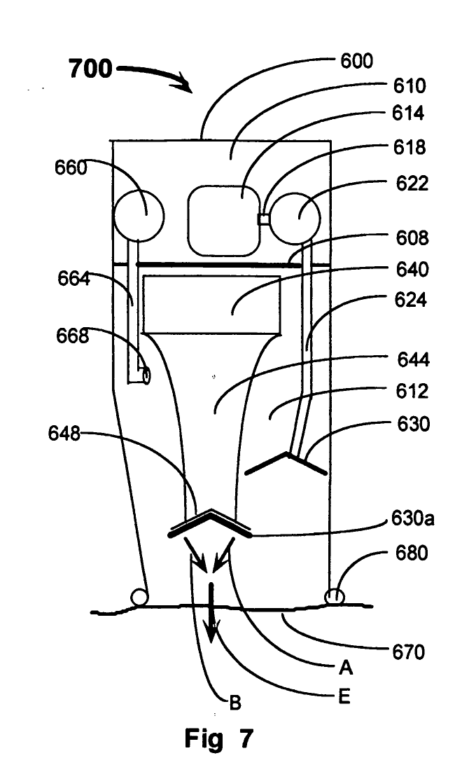

FIG. 7 schematically describes an example of delivery device 700, encased in housing 600 which is attached to biological component 670 via suction rubber 680. Control unit, generating and amplifying elements of the system are not shown. Transducer 640, might be in housing 600, yet might be also located elsewhere, with a wave-guide for transferring the movements to the treatment device, subject of this schematic drawing. Septum 608 separated the device to normal pressure zone 610 and low pressure zone 612, whereas low pressure is created via suction activity employed by pump 660 via opening 668 and tube 664. Delivered substance might be in encapsulated as upside v-shaped 630, and brought from reservoir 614 via guiding element 614, and motor 622, utilizing arm 624.

During operation, mechanical signal given by the transducer, is amplified in amplitude and acceleration rate in tip 644. Maximal, or at least optimal, acceleration rate is achieved in upside v-shaped tip end 648. Substance 630a, or its components, located attached to tip-end 648, are accelerated in the general direction vectors schematically described as arrows A and B. The vectors created, are being further united and amplified in the general direction of schematic delivery vector E, through surface 670 into the biological component.

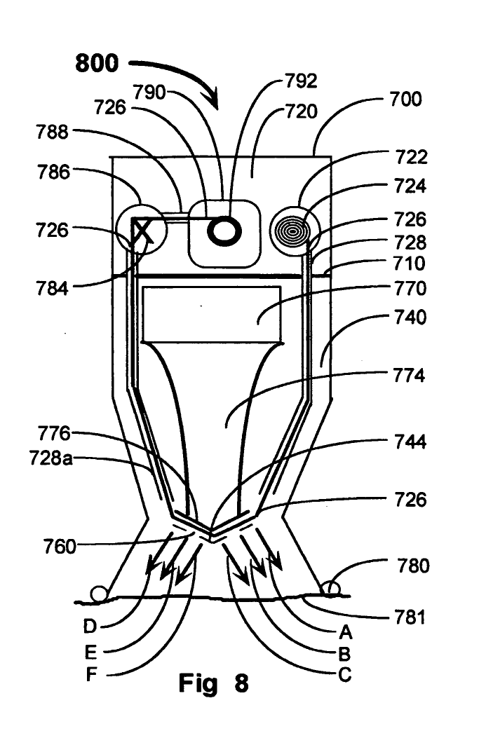

FIG. 8 describes delivery device 800, encased in housing 700 which is attached to biological component surface 781 via rubber ring 780. Septum 710, divides it to space 720 and space 740, whereas space 740 is preferably having slight vacuum. The accelerating elements of the device is composed of transducer 770, guiding tip element 774 and distal v-shaped end 776, having the appropriate acceleration rate. Substance is in a strip form. Bulk of substance 724, is located in sub-encasing 722, from where strip 726 is supplied via channel 728. At least one side of strip 726 contains the substance to be delivered. Strip is forwarded via the space between v-shaped distal end 776 and v-shaped lattice 744, and further via channel 728a. The supply of the strip is carried out by pulling activity, performed by motor 784 in casing 786. It pulls the strip from reservoir 724, as herein above described and further via tube 788 to reservoir 792 of substance depleted strip, in sub-housing 790.

During operation, strip is moving from reservoir 724 to reservoir 792, partially along accelerating v-shaped end 776, and substance is accelerated via openings 760 of lattice 744, in the general direction of arrows A,B,C,D,E and F towards and into surface 781. Operation can be performed in continuous mode, for instance continuous movement of strip together with continuous activation of accelerating element. Operation can be done also in synchronized mode, for instance movement of strip, activation of acceleration, cessation of activation, movement of strip and so on. Combined mode might be also performed.

According to a non limiting embodiment, substances attached to strip are inert solid crystals. Their acceleration at certain angle and acceleration rate towards biological component, will cause during impingement energetic impact on surface of biological component and removal of sub components or layers therefrom. Said debris can be further removed, for instance by a suction activity.

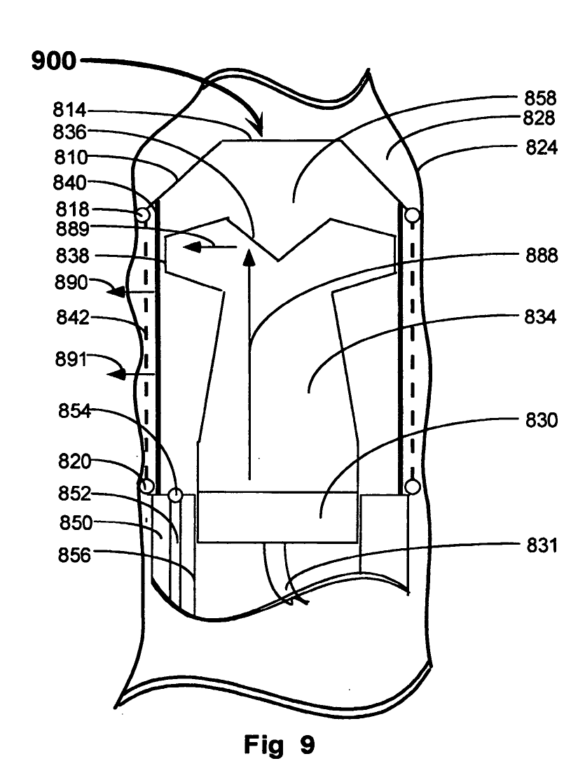

FIG. 9 schematically describes lateral delivery device 900, the delivery component of delivery system. The device might be cylindrical, encased in cylindrical housing 810 having narrow leading edge 814. The device 900 according to this non-limiting example is located in lumen 828 of tube-like biological component 824 which might be for instance be vagina or the coronary blood vessels. Leading edge 814, which essentially is narrow then at least part of other components of the device, widened biological component while being inserted to it, and the biological component is then supported and clasped on the area between rings 818 and 820.

The accelerating element is transducer 830, receiving the electrical signal via cable 831 to create transmission of waves and acceleration of movement. Acceleration of movement is increased via wave guide tip 834. The general direction of propagation of stimuli is from the transducer 830, via wave guide tip in the general direction of schematic arrow 888, reflected from wall 836 in the general direction of schematic arrow 889 and till edge 838 having maximal amplitude and maximal acceleration rate. The surface of the device between rings 818 and 820 is composed of cylindrical lattice cover 842, and inner to it cylindrical reservoir sheet 840 that contain the substance to be delivered. Said substance might for instance be vaccine for local immunization or the vaginal epithelium, localized immune suppression before introducing an IUD, or substance for after-widening stabilization of the coronary arteries, similar to stents, or compounds for paving the coronary arteries before implantation of stents.

After the device reaches its place, certain reduction of the atmospheric pressure in space 858 is created, by suction activity via opening 854 of suction tube 852 of guiding element 850. Stimuli is then created in the transducer, waves are emitted so that edge 838 is accelerated. The acceleration causes delivery of substance from reservoir sheet 840 via opening of lattice 842 and into biological component 824 in the general direction of schematic arrows 890 and 891. The device might be operated also without lattice 842, providing that a certain space can be kept between reservoir 840 and biological component 824. Said space shall preferably be composed of low density medium.

At times, the delivery device might be operated in such synchronization that substances delivered in a circular way, for instance in direction of arrow 890, will get harder after delivery for the creation of a solid ring for mechanical support. That way several rings, with possible supportive linking elements, or any other shape performed according to the lattice design and construction, might be created for establishing for instance a new type of in-situ constructed stent for the stabilization of coronary blood vessels, urethra and other vessels.

During operation, or in synchronic manner, the accelerating device is pulled backwards where transducer 830 is guided along inner wall 856 of guiding element 850. That way each time is affects and delivers substance from another area of reservoir sheet 840. According to non limiting embodiment, the transducer is located outside the delivery device, closer to the other system component such as signal generator, control panel or suction pump, and only appropriate wave guide is located in the device to create the delivery.

It shall be appreciated that also here same device can be used initially to remove portion of tissue, suction for removal of debris, and subsequently the delivery of for instance substances for mechanical support such as for coronary stent or for immunization and the like. The control unit can for example monitor and determine gas pressure in the delivery device, amplitude of vibration, frequency, pulse duration, duty cycle of emitted waves, movement of accelerating element in relation to the biological component or to the supplied substance, rate of supplying the substance and other parameters that might be relevant.

The description and drawings were given for illustrative and non limiting purposes only. The invention embraces any and all modifications, alternatives or rearrangements of the method and device as defined by the claims, including the use of method and device for non-biological components.

1. A method for the administration of substances to and/or through biological components, comprising exposing the substance to be delivered to a cyclic high impact accelerating movement over a short displacement of amplitude, causing acceleration of the substance, rapid displacement of the substance and driving the substance to and/or through biological components, while isolating the bulk of the targeted biological components from the driving force during delivery.

2. A method according to claim 1, wherein the biological components include membranes, organelles, cells, tissues, organs, biological vectors, creatures or artificial biological components including implants and encapsulated cells.

3. A method according to claim 1, wherein the frequency of the cyclic stimulus is at least 1 Hz.

4. A method according to claim 3, wherein the cyclic stimulus is performed by means capable of performing such cyclic movement stimulus.

5. A method according to claim 4, wherein the stimulus is emitted by ultrasound member and delivery is performed by accelerating substance attached to ultrasonic vibrating element.

6. A method according to claim 5, wherein the ultrasound emitting device is composed of piezoelectric crystals.

7. A method according to claim 3, wherein the frequency of the stimulus is 10 Hz to 30 MHz.

8. A method according to claim 7, wherein the frequency of the stimulus is 10 kHz to 3 MHz.

9. A method according to claim 8, wherein the frequency of the stimulus is 20 kHz to 100 kHz.

10. A method according to claim 1, wherein the accelerating movement is for a time period equivalent to at least a quarter of a cycle and at least one stimulus is being given, wherein the substance to be administered is coupled to the stimulating element during at least part of the stimulus period and essentially till desired delivery is carried out.

11. A method according to claim 1, wherein the displacement amplitude is 1 micron to 10,000 microns.

12. A method according to claims 11, wherein the displacement amplitude is 20 microns to 200 microns.

13. A method according to claim 1, wherein the intensity of the stimulus is 0.0001 W/cm² to 10,000 W/cm².

14. A method according to claim 13, wherein the intensity of the stimulus is 0.1 W/Cm² to 100 W/Cm².

15. A method according to claim 14, wherein the intensity of the stimulus is 3 W/cm² to 50 W/cm².

16. The method of claim 1, wherein during delivery a distance exists between the accelerating substance to be delivered and biological component.

17. A method according to claim 16, wherein the space at the distance between accelerating substance to be delivered and biological component, the space through which delivery is performed, is composed of low density medium.

18. A method according to claim 17, wherein the low density medium is gas.

19. A method according to claim 18, wherein reduction of the gas atmospheric pressure is induced in space between substance to be delivered and biological component.

20. The method of claim 19, wherein reduction of the gas pressure is performed by suction.

21. A method according to claim 1, wherein the accelerated substances are delivered without essentially causing any irreversible damage to the bulk of said biological components.

22. A method according to claim 1, wherein the accelerated substances are delivered while causing damage to the superficial zone of biological components.

23. A method according to claim 22, wherein damage is performed to epithelial tissues, which might be moist or keratinized epithelial tissues.

24. A method according to claim 23, wherein damage is utilized for removal of layers of cells or extra cellular matrix.

25. A method according to claim 1, wherein delivered substances are adhered to biological component surface, causing paving-like effect.

26. A method according to claim 1, wherein the accelerated substances are delivered and subsequently within a time period in which at least a portion of said substances remain in biological component target, a controlled damage is caused to at least part of the biological components.

27. A method according to claim 1, used for therapeutic and cosmetic purposes, as well as for diagnostic and experimental purposes, according to the type of substance to be delivered, and the relevant biological component.

28. A method according to claim 27 wherein delivered substances are active agents used for treatment, prevention including active or passive vaccination, creating toxic effect or controlled degeneration by themselves or after activation, initiation or cessation of processes, being substrate elements or resources for procedures, reduction or acceleration of processes, reduction the tension of muscles for instance, sedation or local anesthesia, changing mechanical or chemical properties, giving mechanical support, paving, adding or removing active components, performing genetic manipulations or gene therapy, fertilization, carrying other substances, slow release, staining or marking and the like.

29. A method according to claim 27, wherein delivered substances are essentially inert compounds.

30. A method of claim 27, wherein the substance to be delivered is present in appropriate medium that might be liquid, gel, paste, powder, pellet, solid strip, sleeve and the like.

31. A method of claim 27, wherein the substance require further activation after being delivered.

32. The method of claim 27, wherein the compounds to be administered are soluble.

33. The method of claim 27, wherein the compounds to be administered are complex particles.

34. A method according to claim 33, wherein the complex particles are particles affecting chemical, physiological, or mechanical properties of biological component, wherein particles are essentially selected from the following groups consisting of: Particles impregnated with sedation, tension removal or anti ageing agents, medicaments including radio-active compounds, nutrients, gene-therapy compounds; Particles impregnated with compounds that shall be further activated; Particles having particular coat which might be protective, Particles capable of slow release; Plasmids or other biological or non biological carriers or vectors; Nano-machines, Particles which are biological components such as nucleus, bacteria, viruses or virions, fungi, protozoa, parasites or their fragments.

35. A method according to claim 33 wherein the complex particles are inert particles.

36. A method according to claim 27, wherein more than one substance is delivered to biological compound.

37. A method according to claim 36, wherein different substances are delivered under different acceleration parameters.

38. A method according to claim 36, wherein different substances are delivered under same delivery regime.

39. A method according to claim 1, wherein the delivery is the main treatment.

40. A method according to claim 1, wherein pre-treatment is applied to the biological component before delivery.

41. A method according to claim 40, wherein the delivery method of the present invention is used as pre-treatment method.

42. A method according to claim 1, wherein post-treatment is applied to the biological component after delivery.

43. A method according to claim 42, wherein the delivery method of the present invention is used as post treatment method.

44. A method according to claim 1, wherein the delivery method composed at least part of the steps of any of pre-treatment, treatment, or post treatment, and any combination thereof.

45. A method according to claim 44, wherein at least part of the steps are carried out simultaneously.

46. A method according to claim 44, wherein at least part of the steps are carried out in subsequent order.

47. A method according to claim 1, wherein delivery is performed as stand alone procedure.

48. A method according to claim 1, wherein acceleration element of this invention is accommodated in another housing capable of delivery, to enhance delivery of said another housing while working in combined mode.

49. A method according to claim 48, wherein another housing capable of delivery is gas spring actuating jet injector.

50. A device and a system for use in the method of any one of the preceding claims.

51. A device and a system according to claim 50, substantially as hereinbefore described.

52. A device for the administration by acceleration of substances to and/or through biological components comprising: at least one acceleration agent capable of giving acceleration stimulus and at least a portion of substance to be delivered.

53. A system according to claim 51 containing also means for supply of substance to be delivered to site of acceleration, so to be delivered.

54. A system according to claim 51 containing means for transmitting the acceleration element to the substance.

55. A system according to claim 51, wherein space exists between substance to be delivered and biological component.

56. A system according to claim 55 comprising also means for reduction of the density of material in space between substance to be delivered and biological components

57. A system according to claim 56 whereas means are composed of suction element for removing components and for the reduction of the atmospheric pressure in said space between substance to be delivered and biological component

58. A system according to claim 51 wherein the acceleration agent is comprised of an element capable of emitting ultrasound waves and the system generally comprises power source, control unit, a signal generator, a signal amplifier, a matching unit, at least one transducer capable of emitting ultrasonic waves, and substance.

59. A system according to claim 58 wherein wave-guide exists between the emitting element and the substance to be delivered.

60. A system according to claim 59 wherein said wave-guide amplifies amplitude of vibration, therefore amplifies acceleration rate.

61. A system according to claim 60 wherein wave-guide oscillates at resonance.

62. A system according to claim 59 wherein substance to be delivered composed at least portion of the wave-guide.

63. A system according to claim 51 containing housing encasing at least portion of accelerating element and at least portion of substance to be delivered.

64. A system according to claim 63 wherein housing is attached to affect biological component, using attachment means.

65. A system according to claim 64 wherein said housing is a hand held device.

66. A system according to claim 51 wherein system includes invasive agents such as laparoscope element or catheter.

67. A system according to claim 58 comprising also a reflector for reflecting and changing direction of ultrasonic irradiation.

68. A system according to claim 52 wherein surface of accelerating agent is flat.

69. A system according to claim 68 wherein surface of accelerating agent is shaped otherwise.

70. A system according to claim 52 wherein surface of accelerating agent is covered by compounds capable of reducing the surface tension, therefore enabling easy release of substance to be delivered.

71. A system according to claim 51, wherein delivery device of this invention is attached to, or composed part of, another housing capable of delivery to enhance performance of said another housing.

72. A system according to claim 71, wherein another housing is gas spring actuating jet injector.

73. A system and method as hereinabove described for the treatment of non-biological components.
